(19) United States
(12) Patent Application Publication
(10) Pub. No.: US 2012/0058165 A1
(43) Pub. Date: Mar. 8, 2012

(54) Opacifying Lotion

(76) Inventors: Thomas James Klofta, Cincinnati, OH (US); Kristin Hofmann Miller, Springboro, OH (US); Christoph Hans Peter Bonauer, Burghausen an der Salzach (DE); Samir M. Aziz, Cincinnati, OH (US); Astrid Annette Sheehan, Cincinnati, OH (US)

(21) Appl. No.: 13/220,982

(22) Filed: Aug. 30, 2011

Related U.S. Application Data
(60) Provisional application No. 61/378,096, filed on Aug. 30, 2010.

(51) Int. Cl. A61K 8/02; A61K 8/73; A61K 8/92; A61K 8/29; A61K 8/27; A61K 8/26; A61K 8/25; A61K 8/23; A61K 8/19; A61K 8/81; C11D 3/60; C11D 17/00; A61Q 19/00; A61Q 19/10; A61K 8/84

(52) U.S. Cl. 424/401; 514/770; 514/769; 514/772.3; 514/781; 514/782; 514/783; 510/130; 510/141; 510/445; 510/473; 510/470; 510/475; 510/474; 510/507; 510/508; 510/511; 510/108

(57) ABSTRACT

A lotion for opacifying a wet wipe. A wet wipe may comprise a lotion, the lotion comprising an opacifying agent. The lotion or the substrate may further comprise a retention aid. The substrate may comprise an opacifying agent the same as or different than the opacifying agent of the lotion. A method for opacifying a wet wipe by loading onto a wipe substrate a lotion comprising an opacifying agent. A method for aesthetically modifying a wet wipe by selectively loading onto a wipe substrate a lotion comprising an opacifying agent.

### CROSS REFERENCE TO RELATED APPLICATION

This application claims the benefit of U.S. Provisional Application Nos. 61/378,096, filed on Aug. 30, 2010, the substance of which is incorporated herein by reference.

### FIELD OF THE INVENTION

The disclosure relates to a liquid or semi-liquid composition, useful, for example, for wetting a substrate to form a wet wipe for personal cleansing, the liquid or semi-liquid composition containing organic or inorganic additives to opacify, brighten, or color the wipe-substrate combination.

### BACKGROUND OF THE INVENTION

Wet wipes may be useful for cleaning hard and soft surfaces. Wet wipes may also be useful for delivering functional materials to a surface. For example, a wet wipe may provide skin benefits, such as sunscreen protection, or protection from or treatment of diaper rash. Wet wipes may comprise a substrate, generally a nonwoven material of relatively low basis weight, and a lotion or wetting fluid. The lotion may be aqueous, or, in other cases may contain high quantities of hydrophobic materials. The lotion may comprise cleaning agents suitable for use on a variety of surfaces, including, for example, skin, wood, or countertops. For personal cleansing wipes for use on skin, the lotion may comprise surfactants, emollients, emulsifiers, skin care agents, pH buffers, solvents, particles, preservatives, or other additives for cleaning and/or treating the skin.

Wet wipes may be disposable. That is, they may be intended to be used for one relatively short interval, perhaps minutes, generally less than 3 hours, to clean one or more surfaces, such as one or more nearby countertops, or the skin of the face and neck, or the skin of the perineum and buttocks, and then discarded. A disposable wet wipe may not be intended to be laundered, or otherwise reconditioned or repaired for reuse. It may be desirable to minimize the amount of material in a disposable wet wipe. Reducing the amount of material in a disposable article may reduce the cost of the article, and may reduce the weight and/or bulk of the article. Reducing the weight and/or bulk of an article may have positive environmental impacts, such as reducing the cost of transporting large quantities of the articles during distribution, or reducing the landfill space occupied by discarded articles, or facilitating the degradation (as by composting or recycling) of the article. Accordingly, it would be desirable to produce a wet wipe of minimum weight.

There are at least three ways to reduce the weight of a wet wipe. One approach is to reduce the amount of lotion associated with each wipe. However, a less-wet wet wipe may not clean as effectively as a more-wet wet wipe, or may not be perceived as cleaning as effectively as a more-wet wet wipe.

Another approach is to reduce the overall size of the wet wipe. However, a greater quantity of smaller wet wipes, relative to larger wet wipes, may be needed to complete a cleaning task. Thus, the benefit of having a lower weight wet wipe, or smaller area, may be offset by the increased quantity of wipes consumed for a given task. Further, for messy tasks, a user may prefer a wet wipe of a minimum area, such as an area approximately the size of the user's hand or larger, so that the wipe prevents or reduces direct contact between the mess being cleaned and the user's hand.

A third approach to reducing the weight of a wet wipe is to reduce the basis weight of the wipe, producing a wipe which is relatively low weight for its overall size. However, a wet wipe of relatively low basis weight may be noticeably thin and undesirable for a consumer. In particular, a wet wipe of relatively low basis weight may be less opaque than a wet wipe having a higher basis weight. For some cleaning tasks, such as wiping a baby's bottom during a diaper change, it may be undesirable to see the mess that is being cleaned through the wipe. Further, a wipe which is not opaque may be perceived as being weak, or likely to tear during use, or unable to handle “heavy-duty” cleaning involving rubbing or scrubbing.

Even with relatively high basis weight wet wipes, the substrate of the wipe may be formulated to include materials to opacify the substrate. However, below some minimum basis weight, adding additional opacifiers directly to the substrate materials may be ineffective in increasing the opacity of the wipe. As the basis weight of the wipe is reduced, there may be increased void spaces between the fibers of the wipe. At some point, the void spaces may become large enough such that the mess is not only seen through the wipe, but some kinds of messes, such as liquid, or semi-liquid, or small particulate messes, may be able to traverse and penetrate through the entire thickness of the wipe. If a mess passes through the wipe, the wipe may not effectively “clean” the mess. Further, a mess passed through the wipe may undesirably contaminate the user's hand.

There remains a need for an opaque wet wipe having a low basis weight wipe substrate.

### SUMMARY OF THE INVENTION

In some aspects, this disclosure relates to opacifying lotions, to wet wipes comprising an opacifying lotion, and to methods for opacifying a wet wipe. In some embodiments, an aqueous lotion may comprise a first, inorganic opacifying agent at a level between 0.1% and 2% weight of the inorganic opacifying agent to weight of the lotion. The first, inorganic opacifying agent may have a refractive index greater than 1.33. The first, inorganic opacifying agent may be selected from the group consisting of titanium dioxide, zinc oxide, boron nitride, kaolin clay, calcined kaolin clay, montmorillonite clay, calcined montmorillonite clay, smectite clay, talc, barium sulfate, bentonite clays, silicates (such as sodium magnesium silicates, and fluorosilicates such as sodium magnesium fluorosilicates), silicas including surface modified silicas, calcium carbonate including precipitated calcium carbonate, zirconates such as strontium zirconate, mica coated with titanium dioxide, mica coated with metal oxides, mica coated with titanium dioxide and other metal oxides, and combinations thereof. The aqueous lotion may comprise a retention aid. The retention aid may be selected from the group consisting of acrylamide copolymers, polyethylene imines, copolymers of ethylene imine and acrylamide, polyamines, polyethylene oxides, polydiallyldimethylammonium chloride, hydrogenated castor oil, starches, modified starches, guar gums, modified guar gums, celluloses, carboxymethyl celluloses, modified celluloses, silicas and surface modified silicas, bentonite clays, polyaluminum chloride, and combinations thereof. The retention aid may be present at a level between 0.1% and 20% weight of the retention aid to weight of the lotion.

In some embodiments, a wet wipe may comprise an aqueous lotion and a substrate. The aqueous lotion may comprise a first, inorganic opacifying agent at a level between 0.1% and 2% weight of the inorganic opacifying agent to weight of the lotion. The substrate may have a basis weight less than 75 gsm. The substrate may be a nonwoven. The substrate may comprise fibers. The substrate may comprise a chemical brightener or colorant. The substrate may comprise an opacifying agent (e.g., a second opacifying agent, if the aqueous lotion comprises a first opacifying agent). The substrate may comprise a binder. The second opacifying agent may be a component of the binder. The second opacifying agent may be integral to one or more of the fibers in the substrate. The second opacifying agent in the substrate may be the same as the first, inorganic opacifying agent in the aqueous lotion.

In some embodiments, a method of opacifying a substrate may comprise providing a substrate and loading an aqueous lotion onto the substrate. The aqueous lotion may comprise a first, inorganic opacifying agent at a level between 0.1% and 2% weight of the opacifying agent to weight of the lotion. The substrate may have a basis weight between 20 and 75 gsm. The substrate may comprise a second opacifying agent. The first and second opacifying agents may be the same. The aqueous lotion may be loaded onto the substrate at a level between 110% and 600%, weight of the aqueous lotion to weight of the unloaded substrate. The substrate may be a nonwoven. The substrate may be a nonwoven having a basis weight between 20 and 75 gsm. The aqueous lotion may be a personal cleansing composition. The method may further comprise modifying the substrate. The modification may be selected from the group consisting of hydro-molding, hydro-embossing, ring rolling, structural elongation, consolidation, stretch aperturing, differential elongation, chemical treatment, and thermal treatment. The modification may create densified and undensified or dedensified regions of the substrate. The aqueous lotion may be applied homogeneously to the wipe. The aqueous lotion may be selectively applied to the wipe. The aqueous lotion may be selectively applied to the wipe in a pattern complementary to the densified and undensified or dedensified regions of the substrate.

In some embodiments, an aqueous lotion may comprise a first, polymeric opacifying agent at a level between 0.1% and 10% weight of the polymeric opacifying agent to weight of the aqueous lotion. The first, polymeric opacifying agent may have a refractive index greater than 1.33. The first, polymeric opacifying agent may be selected from the group consisting of styrene/vinyl pyrrolidone copolymers, styrene/acrylic copolymers, styrene/acrylamide copolymers, and combinations thereof. The aqueous lotion may comprise a retention aid. The retention aid may be selected from the group consisting of acrylamide copolymers, polyethylene imines, copolymers of ethylene imine and acrylamide, polyamines, polyethylene oxides, polydiallyldimethylammonium chloride, hydrogenated castor oil, starches, modified starches, guar gums, modified guar gums, celluloses, modified celluloses, silica, bentonite clays, polyaluminum chloride, and combinations thereof. The retention aid may be present at a level between 0.1% and 20% weight of the retention aid to weight of the lotion.

In some embodiments, a wet wipe may comprise a substrate and an aqueous lotion comprising a first, polymeric opacifying agent at a level between 0.1% and 10% weight of the polymeric opacifying agent to weight of the aqueous lotion. The substrate may have a basis weight less than 75 gsm. The substrate may be a nonwoven. The substrate may comprise fibers. The substrate may comprise a chemical brightener or colorant. The substrate may comprise a second opacifying agent. The substrate may comprise a binder. The second opacifying agent may be a component of the binder. The second opacifying agent may be integral to one or more of the fibers in the nonwoven substrate. The second opacifying agent in the substrate may be the same as the first opacifying agent in the aqueous lotion.

In some embodiments, a method of opacifying a substrate may comprise providing a substrate and loading an aqueous lotion onto the substrate. The aqueous lotion may comprise a first, polymeric opacifying agent at a level between 0.1% and 10% weight of the opacifying agent to weight of the aqueous lotion. The substrate may have a basis weight between 20 and 75 gsm. The substrate may comprise a second opacifying agent. The first and second opacifying agents are the same. The aqueous lotion may be loaded onto the substrate at a level between 110% and 600%, weight of the aqueous lotion to weight of the unloaded substrate. The substrate may be a nonwoven substrate having a basis weight between 20 and 75 gsm. The aqueous lotion may be a personal cleansing composition. The method may further comprise modifying the substrate. The modification may be selected from the group consisting of hydro-molding, hydro-embossing, ring rolling, structural elongation, consolidation, stretch aperturing, differential elongation, chemical treatment, and thermal treatment. The modification may create densified and undensified or dedensified regions of the substrate. The aqueous lotion may be applied homogeneously to the wipe. The aqueous lotion may be selectively applied to the wipe. The aqueous lotion may be selectively applied to the wipe in a pattern complementary to the densified and undensified or dedensified regions of the substrate.

In some embodiments, a wet wipe comprises a substrate and an aqueous lotion comprising an opacifier. The substrate may have densified and undensified or dedensified regions. The aqueous lotion may be selectively applied to the substrate in a pattern complementary to at least a portion of the densified and undensified or dedensified regions of the substrate. The wet wipe may comprise a second lotion. The aqueous lotion may be applied to the densified regions of the substrate. The aqueous lotion may be applied to the undensified or dedensified regions of the substrate. The wet wipe may comprise a colorant.

In some embodiments, a wet wipe may comprise a substrate and an aqueous lotion comprising an opacifier. The aqueous lotion may be selectively applied to the substrate to create a visible pattern or design. The wet wipe may comprise a colorant.

In some embodiments, a method of heterogeneously loading a lotion onto a substrate comprises providing a substrate having varied physical or chemical properties in at least one of the x-, y-, and z-directions, providing a lotion adapted to interact with one or more of the physical or chemical properties of the substrate, and applying the lotion to the substrate. At least one of the varied physical or chemical properties of the substrate may be selected from the group consisting of pore size, fiber length, fiber hydrophilicity, fiber hydrophobicity, fiber charge, fiber pH, and combinations thereof. The lotion adaptation may include at least one of the physical or chemical properties selected from the group consisting of charge, pH, presence of a retention aid, type of retention aid, viscosity, hydrophilicity, hydrophobicity, surface tension, and combinations thereof. The substrate may have different fiber types in two or more discrete regions in the x- and y-directions. The substrate may have different fiber types in two or more discrete regions in the z-direction.

### BRIEF DESCRIPTION OF THE DRAWINGS

FIG. 1 is an exemplary, scanned image of an embossed substrate loaded with a conventional lotion.

FIG. 2 is an exemplary, scanned image of an embossed substrate loaded with a lotion comprising 0.6% TiO₂.

### DETAILED DESCRIPTION OF THE INVENTION

As used herein, “aqueous” refers to a composition containing water as its major constituent. An aqueous composition may comprise greater than 50%, or at least 80%, or at least 90%, or more, but less than 100%, water, comparing weight of water to weight of the composition. As used herein, “nonaqueous” refers to a composition containing an oil as its major constituent. A nonaqueous composition may comprise between 0% and 50% water, comparing weight of water to weight of the composition.

As used herein, “basis weight” refers to the weight of a single ply of substrate normalized over its surface area. Basis weight may be expressed as grams per square meter (gsm), and may be measured using the EDANA standard test method #40.3-90.

As used herein, “binder” refers to a substance or composition which is used to create or strengthen bonds between the constituent parts of a wipe substrate. For example, a binder may create or strengthen bonds between individual fibers in a spunbond layer in a wipe substrate comprising a spunbond layer, or to create or strengthen bonds between fibers in adjacent spunbond and meltblown layers in a wipe substrate comprising at least one spunbond layer and at least one meltblown layer. A binder may be, but is not necessarily, an adhesive.

As used herein, “emulsion” refers to a mixture of two or more immiscible liquids held in suspension by small percentages of substances called emulsifiers. Emulsifiers are of many types, for example: (1) Proteins or carbohydrate polymers, which act by coating the surface of the dispersed fat or oil particles, thus preventing them from coalescing (sometimes called protective colloids); and (2) Nonionic emulsifiers like polyglycol ethers (examples include fatty alcohol polyglycol ethers, fatty alcohol polyglycerol ethers, fatty alcohols, ethoxylated fatty alcohols, fatty acid polyglycol esters, fatty acid polyglycerol esters, fatty acid alkanol amides, and ethylene oxide/propylene oxide block polymers) and polyol emulsifiers (examples include glycerine fatty acid esters, sorbitan esters, and alkyl glycosides); (3) Cationic emulsifiers like amine salts, amines with alkyl groups, and quaternary ammonium compounds; (4) Anionic emulsifiers like soaps, alkylbenzene sulfonates, fatty alcohol sulfates, alkane sulfonates, sulfo succinic acid alkyl esters, ether carboxylic acids, and sarcosinates; and (5) Amphoteric emulsifiers like alkyl betaines and sulfo betaines and mixtures of the classes noted above. Emulsifiers are able to reduce surface tension to decrease the thermodynamic driving force leading to coalescence. The emulsifiers may also provide a physical barrier at the interface to aid in inhibiting coalescence. All emulsions consist of a continuous phase and a disperse phase: in an oil-in-water (o/w) emulsion, such as milk, water is the continuous phase and butterfat (oil) is the dispersed phase; in a water-in-oil (w/o) emulsion, such as butter, free fat (from crushed fat globules) is the continuous phase and unbroken fat globules plus water droplets make up the dispersed phase. An emulsion is herein described as having an oil component or an oil phase, however, it should be understood that the lipophilic or “oil” component of an oil-in-water emulsion may include solids or semi-solids, such as waxes, jellies, gels, particles, and the like.

An emulsion may be stable from a kinetic standpoint, but never from a thermodynamic standpoint. The emulsion may be stable over the course of its making until it is applied to the wipe substrate. The stability of emulsions can be measured using a TurbiscanLab instrument from the company named Formulaction (L'Union, Toulouse), or equivalent, according to the manufacturer's instructions. The TurbiscanLab can detect emulsion instability by measuring differences in the backscattered light flux for regions between the top and bottom of the sample cell in which the emulsion is contained. For solid opacifying agents of densities different than that of the liquid phase, the solid opacifying agents may also remain homogeneous in the liquid phase prior to addition to the dry substrate. The homogeneity of the solid opacifying agents within the liquid lotion matrix can also be measured with the TurbiscanLab instrument, or equivalent, according to the manufacturer's instructions. It should be appreciated that both the emulsion and opacity particle suspension can be stabilized (the kinetics slowed down), for example, through the use of emulsifiers, wetting agents, rheological agents, dispersing agents, density, particle size and shape, or combinations thereof.

As used herein, “lotion loading” refers to the process of applying a lotion to a substrate to form a wet wipe. A “loaded” substrate is associated with a lotion. An “unloaded” or “dry” substrate has not been treated with a lotion. It should be understood that a “dry” substrate may have a significant water or fluid content, as from liquid retained by the substrate during substrate formation and/or processing, or from liquid absorbed by the substrate, as from ambient humidity.

As used herein, “opacifying” or “increasing the opacity of” an object means modifying the object in a manner which increases the opacity of the object as measured using the test method for opacity described below. The terms “opacifying” and “increasing the opacity of” can be contrasted with creating an impression of opacity or creating an impression of increased opacity, which might or might not correspond to an objective, measurable increase in opacity. In some embodiments, an opacified object may be at least 5%, or at least 10%, or at least 20% more opaque than the object as measured before it was modified.

As used herein, “regions” or “portions” refer to an incomplete part of the surface or depth of a substrate, that is, less than 100% of a given surface (top or bottom) of a substrate, or less than 100% of the depth of a substrate. A “region” or “portion” may be located anywhere along the length, width, or depth of a substrate. A “region” or “portion”, as used herein, should be understood to be macroscopic, and, more particularly, to be visible to the unaided human eye. That is, a “region” or “portion” may have no dimension smaller than 0.05 mm, when measured at the widest point along a given direction. For example, a “region” may be a circular dot having a radius of at least 0.025 mm, or an ellipse having a minor axis diameter of at least 0.05 mm, or a rectangle extending at least 0.05 mm in each of the x- and y-directions.

As used herein, “suspension” refers to a system in which very small particles (solid, semisolid, or liquid) are more or less uniformly dispersed in a liquid medium. If the particles are small enough to pass through filter membranes, the system is a colloidal suspension (or solution). Examples of solid-in-liquid suspensions are comminuted wood pulp in water, which becomes paper on filtration and drying; the fat particles in milk; and the red corpuscles in blood.

As used herein, “retention aid” refers to a substance or composition which is used to create or increase an affiliation between a wipe substrate and an opacifier (or brightener, or colorant, as discussed below), such that the opacifier, brightener, or colorant is less likely to transfer to a surface when the wipe is swiped or rubbed across the surface. A retention aid may be applied to the substrate or incorporated into a wetting lotion. The same substance or composition may serve as a binder and a retention aid, or different substances or compositions may be used if both a binder and a retention aid are desired. The retention of the opacifier, brightener, or colorant can be measured through the use of a Sutherland Rub Tester manufactured by KVP Sutherland Paper Company of Kalamazoo, Mich., or equivalent, according to the manufacturer's instructions.

As used herein, percentages are given as the weight of the component to the total weight of the lotion, unless otherwise indicated. Percentages reflect 100% active component material. For example, if a component is available in a dispersion at a concentration of 50% component to dispersion, by weight, twice as much of the dispersion, by weight, would be added to the lotion to provide the equivalent of 100% active component.

As described above, previous approaches to opacifying wet wipes include adding opacifiers to the substrate. A new approach to opacifying or brightening a wet wipe is to opacify the wetting lotion rather than the substrate. This approach may facilitate the opacification or brightening of wipes even when the basis weight is below a threshold level at which adding opacifiers or fillers to the substrate no longer compensates for the light-transmitting spaces between fibers or components of the substrate. In some embodiments, opacifiers or other particulates in the lotion may physically occlude some of the spaces between the fibers or components of the substrate, such that bleed-through of messes through the wipe is reduced. In some embodiments, the incorporation of particulate opacifiers, brighteners, or colorants into the wetting lotion may contribute to improved cleaning performance. In some embodiments, the opacifier, brightener, or colorant may provide skin benefits, such as rash healing benefits, reduced friction between the wipe and surface upon which it is used, improved cleaning, or protection from damaging ultra-violet radiation from the sun. Opacifying, brightening, or coloring the lotion rather than the substrate may be more economical (i.e., less expensive) than increasing the basis weight of the wipe substrate.

A wetting lotion may function primarily to provide moisture to a wipe, or may contain other active ingredients, such as surfactants, emulsifiers, emollients, film-formers, anti-stick agents, preservatives, pH buffers, rheology modifiers, and the like, as described, for example, in U.S. Pat. No. 7,666,827, U.S. Pat. No. 7,005,557, U.S. Patent Application Publication No. 2007/0286894, and U.S. patent application Ser. No. 12/771,391. A wetting lotion may further comprise an opacifier, brightener, colorant, or mixtures thereof. The opacifier may have a refractive index of at least 1.33. The wetting lotion may be aqueous or nonaqueous.

Some exemplary opacifiers include polymeric materials like styrene/vinyl pyrrolidone copolymers, styrene/acrylic copolymers, and styrene/acrylamide copolymers. Suitable, commercially available, polymeric particulates include, but are not limited to, styrene/acrylic copolymers available from ROHM & HAAS and styrene/vinyl pyrrolidone copolymers available from ISP. Other opacifiers may include inorganic materials, such as titanium dioxide, zinc oxide, kaolin clay, calcined kaolin clay, montmorillonite clay, calcined montmorillonite clay, talc, barium sulfate, bentonite clays, silicates, silicas, calcium carbonate, precipitated calcium carbonate, zirconates such as strontium zirconate, and mica substrates coated with titanium dioxide and/or metal oxides like iron oxide or tin oxide. Mixtures of different opacifiers, including mixtures of polymeric opacifiers, inorganic opacifiers, or polymeric opacifiers and inorganic opacifiers, may be used.

The opacifier may be added to the wetting lotion as particulates or as particles predispersed in a liquid mixture. Generally, particulates having irregular surfaces will have a higher refractive index than smoother, rounder, or more spherical particles. However, irregular particulate surfaces may contribute to a coarse or gritty texture.

For skin, a coarse or gritty texture might be acceptable as an exfoliating wipe. A coarse or gritty texture may also be acceptable, or even preferred, in a cleaning wipe for inanimate surfaces, such as bathroom fixtures, floors or countertops. In other instances, a coarse or gritty texture may be undesirable. For example, coarseness or grittiness may be tolerated or desired in a skin-cleansing wipe intended for heavy-duty cleaning or exfoliation, because a gritty texture may be associated with better cleaning, but relatively low levels of coarseness or grittiness may be unacceptable in a skin-cleansing wipe intended for use on delicate skin, such as facial and neck skin, or the perineum, or infant or toddler skin, or the perineum of an infant or toddler. Particulate selection, for both polymeric materials and the other opacifiers described herein, may therefore require a product-specific balance between irregular, light-scattering surface characteristics versus rounded, smooth surface characteristics for delivering a smooth tactile lotion feel. Particle size may also contribute to a smoother tactile feel. Particle sizes under 50 microns may help achieve a non-gritty tactile feel. Particle concentration may also be adjusted for tactile feel. Higher concentrations, such as particle concentrations between 10% and 20% or greater, may be associated with grittier products, and lower concentrations, such as particle concentrations less than 5%, may be associated with smoother products.

In some embodiments, particles having irregular surfaces may be treated with a softening aid to improve the tactile feel of the irregular surface. Some softening aids include, but are not limited to, mineral oil, vegetable oils, dimethicone, functionalized silicones, or combinations thereof. In some cases, the surface treatment may be covalently bound to the particle surface. Some covalently bound softening aids may also increase the dispersibility of the particle in a liquid matrix, including aqueous matrices.

One concern with adding an opacifying agent to a wetting lotion is that the opacifier from the lotion may transfer to the surface on which the wipe is being used. For some wipe products, it may be undesirable for the wetting lotion to leave a visible residue. For example, it may be undesirable for a wet wipe to leave a visible residue on surfaces, including skin, clothing, or other textiles. As a more particular example, it may be undesirable to transfer a white opacifier to a darkly colored textile (e.g., a textile in saturated shades of black, brown, blue, or the like), which may make the textile seem dusty, dirty, faded, or otherwise inferior to its uncontaminated appearance. In some embodiments, the concentration of the opacifier in the lotion may be between 0.1% and 10%, or between 0.3% and 2%, by weight of the opacifier to weight of the lotion, to provide some opacity while minimizing the amount of visible particulate likely to be transferred to a surface while using the wet wipe. The desirable level of opacifier in the lotion may vary based on the type of opacifier used. Some transparent and opacifying organic plastics, for example, may be acceptable up to 10%, or at concentrations greater than 10%, as they may be relatively less noticeable if they do transfer to a surface. Some inorganic particles, such as zinc oxide or titanium dioxide, may be relatively more noticeable if they do transfer to a surface, and, therefore, may be acceptable at lower concentrations, such as concentrations of less than 5%, or less than 2%.

In some embodiments, the lotion further comprises a retention aid. The retention aid may function to increase the affinity of the opacifier for the wet wipe substrate, to reduce the likelihood that opacifiers from the lotion will transfer to a surface while using the wipe. A retention aid may be added to the wipe substrate. That is, a retention aid may be formulated into the fibers or components of the substrate before the substrate is formed; or may be added to the substrate during formation, for example, by adding particulates of the retention aid as the substrate is formed, for example, by spunbonding, meltblowing, airlaying, carding, and the like; or the retention aid may be added to the formed substrate prior to loading a wetting lotion onto the substrate. For example, the retention aid may be formulated into a liquid solution and applied to the substrate prior to the application of the lotion. As another example, the wipe lotion could be applied before the retention aid liquid composition. As yet another example, the wipe lotion and the retention aid liquid composition could be combined just prior to application to the substrate. The retention aid may be added to the lotion, before, during, or after the addition of the opacifier to the lotion. The retention aid may be combined with the opacifier, and the combined retention aid-opacifier may be added to the lotion.

The selection of a retention aid may be guided by the chemistry and characteristics of the substrate and/or the opacifier. For example, if a 100% polypropylene substrate having no net charge is used, a retention aid having a lipophilic moiety might be used to help associate the retention aid with the non-polar polypropylene surface. To associate this retention aid with the opacifier, the retention aid may, in addition to the lipophilic moiety to associate the retention aid with the polypropylene, comprise a functional group capable of associating with the opacifier. In this way, the retention aid acts as a bridge by associating with both the substrate and the opacifier. As another example, if a 100% cellulose substrate modified to have a net negative charge is used, a retention aid having at least a moiety with a net positive charge might be used to help associate the retention aid with the negatively charged cellulose surface.

In some embodiments, it is not necessary for the retention aid to form a chemical bond with the substrate and/or the opacifier, although some retention aids may form one or more chemical bonds with the substrate and/or the opacifier. There are a variety of chemical bonding types that can occur between the retention aid and substrate, the retention aid and opacifier, opacifier and substrate, and all other permutations of association. These bond types include covalent bonds; bonds formed via Van der waals forces; hydrogen bonds; ionic bonds; ionic attractions, such as colloidal interactions; and combinations of these various bonding types. The retention aid may associate with the opacifier and the substrate via different chemical bonding mechanisms.

In some embodiments, the retention aid associates with the opacifier and/or the substrate via physical interaction. For example, the retention aid may be sized and applied to the substrate such that the retention aid is physically entrapped in the substrate. One exemplary method of entrapping retention aids in a substrate is filtering a solution of appropriately sized retention aids through the substrate. The opacifier may be retained by mechanical entrapment in the structure made up by the fibers of the substrate. For example, the opacifying particle may be retained in the substrate fiber network through interaction with mechanical imperfections on the substrate fiber walls; or through capillary attachment of the opacifier within either the pores made up by the fibers of the substrate or within the channels along a fiber surface; or through mechanical attraction via diffusion of the opacifying agent into the lumen of the fiber. Thus, retention of the opacifier within the substrate may occur via chemical or mechanical mechanisms, or combinations thereof.

A mixture of different retention aids may be helpful, for example, in embodiments comprising a mixture of different opacifiers, or in embodiments comprising a mixture of different substrate fiber chemistries. Of course, the substrate may be varied to accommodate a preferred opacifier and/or retention aid, or the opacifier may be varied to accommodate a preferred substrate and/or retention aid.

Another consideration in selecting combinations of substrates, opacifiers, and/or retention aids may be the pH of the lotion. The pH of the lotion may cause the opacifier charge to be opposite of that of the substrate such that retention is enhanced via attraction of the opposite charges. Inorganic and organic surface treatments of the substrate may also be used to help retain the opacifier on or in the substrate. For example, alum may be added to pulp fibers at a pH of approximately 6 to make the pulp fibers positively charged. This aids in the retention of opacifiers like talc or clay, which are negatively charged at a pH of 6.

Exemplary retention aids include, but are not limited to, polyelectrolytes of the nonionic, anionic, cationic, or amphoteric types; polysaccharides; galactomannans; inorganic salts and particles; or combinations thereof, selected, as described above, for compatibility with a specific substrate and opacifier. Exemplary polyelectrolytes include, but are not limited to, polyamideamine, polyacrylamides, acrylamide copolymers, polyethylene imines, copolymers of ethylene imine and acrylamide, polyamines, polyethylene oxides, and polydiallyldimethylammonium chloride. Exemplary polysaccharide materials include, but are not limited to, starches, cationic starch and other modified starches, cellulose and modified celluloses, and chitin and modified chitins. Exemplary galactomannans include, but are not limited to, guar gums, modified guar gums, locust bean gum, and modified locust bean gums. Exemplary inorganic salts and particles include, but are not limited to, alum, silica, bentonite clays, and polyaluminum chloride. Mixtures of different retention aids, including mixtures of different types of retention aids (such as polyelectrolytes, polysaccharides, galactomannans, and inorganic salts and particles) may be used.

Some materials, such as bentonite clays, may serve as opacifiers and may also serve as retention aids. In some embodiments, the opacifier and the retention aid are the same. In some embodiments, the opacifier and the retention aid are the same or similar chemical entities, but in different sizes, shapes, or distributions on the wipe. In some embodiments, the opacifier and the retention aid are different. The retention aid, if used, may be present in the lotion at a level between 0.1% and 20%, weight of the retention aid to the total weight of the lotion.

In some embodiments, the lotion may comprise an emulsion. Some oil-in-water emulsions may serve as opacifiers. For example, some emulsions may have an opaque, white appearance. The opaque, white appearance is due to refractive index differences between the oil and water components of the emulsion. Opaque emulsions may also comprise dispersed droplets having a relatively large particle size of a wide distribution. For example, “large” oil droplets in the size range of 0.1 to 10 microns may cause light scattering of many different wavelengths in many different directions. This is known as the Tyndall effect. Thus, an emulsion may appear white due to a large droplet size (relative to the visible wavelengths of light) of a wide particle size distribution, such that the droplets are capable of scattering many wavelengths of incident light in many different directions. Particle size and particle size distribution are influenced by both the chemistry of the emulsion (i.e., the relative amounts of oil and liquid; the kind(s) of oils in the emulsion; and the presence of other compounds in the aqueous component, such as emulsifiers and surfactants) and the process used to make the emulsion. Particle size distribution is also influenced by processing techniques and parameters. For example, high shear processing tends to produce small droplets of oil dispersed in the water phase, the small droplets having a size of around 0.1 microns, which reflect light having ultraviolet (UV) wavelengths (UV wavelengths are not visible to the unaided human eye). Such emulsions tend to be transparent. Low-energy, low-shear processes may facilitate the generation of larger particle sizes, which may reflect visible wavelengths of light to create an opaque, white appearance. Opaque emulsions may be used instead of or in addition to inorganic and/or polymeric particles.

Opacifying emulsions may have a particle size distribution of 0.1 to 10 microns. Some exemplary oleaginous components of an opacifying oil-in-water emulsion include sunflower oil, olive oil, jojoba oil, other vegetable oils, mineral oil, silicone fluids, functionalized silicone emollients, and combinations thereof. Some exemplary low-energy, low-shear processing techniques include paddle mixing or paddle blending. Of course, an opacifying lotion or even an opacifying emulsion may be processed using relatively high-energy and/or high-shear processes, to form particles of smaller size.

Opaque emulsions may not provide the same magnitude of opacity increase, relative to the dry substrate, as other opacifying lotions described herein. For example, the ability to increase the oil concentration or oil droplet size to increase the emulsion opacity may be limited by the need to maintain an aqueous base, or surfactant concentrations, or other lotion properties related to cleaning effectiveness or lotion stability (i.e., maintaining a stable emulsion over time, such that the oil droplets do not separate from the water phase). In embodiments where there is limited flexibility in manipulating the emulsion properties, an opaque emulsion may be used with other opacifiers, brighteners, and/or colorants, as described herein.

The lotion may be loaded onto a substrate. The lotion may be applied such that the lotion application process applies lotion to either one side or both sides of the substrate. A plurality of wipe substrates are known in the art, and disclosed, for example, in U.S. Pat. No. 6,673,358 to Cole et al. and U.S. Patent Publication No. 2007/0286894 to Marsh et al. Disposable wipes are often constructed of nonwoven materials. “Nonwoven” refers herein to a fibrous structure made from an assembly of continuous fibers, coextruded fibers, non-continuous fibers and combinations thereof, without weaving or knitting, by processes such as spunbonding, carding, meltblowing, airlaying, wetlaying, coforming, or other such processes known in the art for such purposes. The fibers may be continuous fibers, staple fibers, or combinations thereof. The process for incorporating a fiber into a substrate may be selected based upon the sorts of component materials used and the desired properties of the substrate web. The nonwoven material may comprise one or more layers of fibrous assemblies, wherein each layer may include continuous fibers, coextruded fibers, non-continuous fibers, and combinations thereof.

A suitable wipe may be constructed of any material or blend of materials which produces suitable flexibility, durability, and, if desired, liquid absorbency. Suitable fibers may be natural, cellulosic, wholly synthetic, or some combination of fibers. Natural or synthetic fibers may be treated or otherwise modified mechanically or chemically to provide desired characteristics or may be in a form that is generally similar to the form in which they can be found in nature.

In certain embodiments, particular combinations of fibers may be selected to provide desired characteristics. The fibers may be of one or more types, including different compositions or shapes of fibers. For example, fibers of certain lengths, widths, coarseness, shape or other characteristics may be combined in certain layers, or in distinct layers separate from each other. In some embodiments, suitable materials include viscose, polypropylene, polypropylene-viscose blends, polyethylene, polyester, rayon, cotton, cellulose, modified cellulose, pulp, and combinations thereof. The fibers may have core-and-sheath construction, and the core and sheath materials may be the same compositions or different compositions. The fibers may have inherent shapes, such as dog-bone, tri-lobal, multi-lobal, rounded, delta. Combinations of fibers having different inherent shapes may be used. References to substrate “fibers”, unless otherwise noted, include substrate components which are not true fibers, such as films, particles, yarns (or other collections of fibers), and the like. That is, a reference to a substrate fiber is not intended to limit the description to nonwoven substrates comprising true fibers.

Multi-lobal fibers may facilitate the creation of capillary channels within the substrate. Capillary channels may increase the amount of lotion drawn into the substrate, away from the surface of the substrate. Lotion residing in the “center” of the substrate—that is, away from the surface of the substrate, although not necessarily at the vertical or horizontal center of the substrate—may increase particle retention when the wipe is used. Movement of the lotion to the center of the substrate may also facilitate the movement of particulates into the interstitial spaces between the fibers of the substrate, and therefore help reduce bleed-through of messes through the interstitial spaces of the wipe during use.

The substrate materials may also be treated to improve the softness and texture thereof. The substrate may be subjected to various treatments, such as, but not limited to, physical treatment, such as hydro-molding, hydro-embossing, ring rolling, as described in U.S. Pat. No. 5,143,679 issued to Weber et al. on Sep. 1, 1992; structural elongation, as described in U.S. Pat. No. 5,518,801 issued to Chappell et al. on May 21, 1996; consolidation, as described in U.S. Pat. Nos. 5,914,084 issued to Benson et al. on Jun. 22, 1999; 6,114,263 issued to Benson et al. on Sep. 5, 2000; 6,129,801 issued to Benson et al. on Oct. 10, 2000 and 6,383,431 issued to Dobrin et al. on May 7, 2002; stretch aperturing, as described in U.S. Pat. Nos. 5,628,097 issued to Benson et al. on May 13, 1997; 5,658,639 issued to Curro et al. on Aug. 19, 1997 and 5,916,661 issued to Benson et al. on Jun. 29, 1999; differential elongation, as described in US Publication No. 2003/0028165A1 published on Feb. 6, 2003 by Curro et al.; and other solid state formation technologies as described in U.S. Publication No. 2004/0131820A1 published on Jul. 8, 2004 by Turner et al. and U.S. Publication No. 2004/0265534A1 published on Dec. 30, 2004 by Curro et al., zone activation, pressure bonding, needlepunching, airlaying, tufting, compaction, and the like; chemical treatment, such as, but not limited to, rendering part or all of the substrate hydrophobic, and/or hydrophilic, or increasing the hydrophobicity or hydrophilicity, and the like; thermal treatment, such as, but not limited to, thermal-embossing, softening of fibers by heating, thermal bonding and the like; and combinations thereof. Without being bound by theory, it is believed that a textured substrate may facilitate removal of bodily exudates or other soils by improving the ability to grip or otherwise lift the soils from the skin during cleansing.

The substrate may have a basis weight between about 15, 30, 40 or 45 grams/m² and about 65 or 75 grams/m². The substrate may have a basis weight less than 75 gsm, or less than 65 gsm. One exemplary substrate may be a carded nonwoven comprising a 40/60 blend of viscose fibers and polypropylene fibers having a basis weight of 58 grams/m² as available from Suominen of Tampere, Finland as FIBRELLA™ 3160. Another exemplary material may be FIBRELLA™ 3100 which is a 62 grams/m² nonwoven web comprising 50% w/w 1.5 denier polypropylene fibers and 50% w/w 1.5 denier viscose fibers. Another suitable material for use as a substrate may be SAWATEX™ 2642 as available from Sandler AG of Schwarzenbach/Salle, Germany. Yet another suitable material for use as a substrate may have a basis weight of from about 40 grams/m² (gsm) to about 200 gsm and have a 20/80 blend of viscose fibers and polypropylene fibers. The substrate may also be a 60/40 blend of pulp and viscose fibers.

In another embodiment, the substrate may be biodegradable. For example the substrate could be made from a biodegradable material such as a polyesteramide, or a high wet strength cellulose. The substrate may also be dispersible, that is, the substrate or designated portions of the product may sufficiently dissolve or disintegrate in water such that the substrate may be discarded in sewer or septic systems without presenting any problems for typical household or municipal sanitization systems. The materials and methods for making such a dispersible substrate are described, for example, in WO 2007/125443 to Kimberly-Clark Worldwide, Inc.; in U.S. Pat. No. 4,755,421 to Manning et al.; in U.S. Pat. No. 7,285,504 to Jones et al.; in U.S. Pat. No. 7,157,389 to Branham et al.; and in U.S. Pat. No. 7,101,612 to Lang et al.

Other suitable substrates include coform substrates, as described in U.S. Pat. No. 4,100,324 to Anderson et al., substrates formed by hydrodynamic needling, as described in U.S. Pat. No. 6,842,953 to Orlandi, and the substrates described in U.S. Pat. No. 7,972,986 to Barnholtz et al.

The substrate may comprise an opacifier. The substrate opacifier, if present, may be the same as or different than the opacifier in the wetting lotion, however, the substrate opacifier will be bound in or to the substrate, such that the substrate opacifier does not transfer from the substrate while using the wipe. For example, the substrate fibers may comprise a polymer, the polymer comprising an opacifier in the polymer melt formulation, such that the opacifier is structurally encapsulated in the substrate fibers. That is, the opacifier may be integral to one or more of the fibers making up the substrate. The substrate may include a binder to maintain cohesion between the fibers of the substrate. Exemplary binders include, but are not limited to, adhesives such as latex-based adhesives, latex, and combinations thereof. The substrate opacifier, if present, may be added to the substrate as part of the binder. Instead of, or in addition to, a “second” opacifier in the substrate (which may, for example, be the third, or fourth, or greater opacifier in the lotion-loaded wipe if, for example, more than one opacifier is used in the lotion or the substrate), the substrate may include a chemical brightener and/or colorant.

A chemical brightener may be used to give the substrate a higher perceived whiteness by absorbing ultraviolet wavelengths of light and emitting blue wavelengths of light. Exemplary brighteners include, but are not limited to, brighteners available as UVITEX or TINOPAL brands from Ciba Specialty Chemicals (Basel, Switzerland). The UVITEX or TINOPAL brand brighteners may be particularly, but not exclusively, effective for incorporation into substrate fibers made from polypropylene. The blue wavelengths emitted by such brighteners may be in the range of approximately 420-470 nm. That is, a brightener may increase the relative proportion of blue light emitted from the surface of the substrate, which increases the perceived whiteness of the wipe. The increased perception of whiteness may itself give an impression of increased wipe opacity, even if the light transmittance through the wipe is not decreased by the chemical brightener. The increased whiteness may also be associated with a “cleaner” or “fresher” looking wipe, before using the wipe.

In some embodiments, a colorant is added to the substrate. For example, the substrate may comprise dyes or pigments which give the substrate a subtle tint of, for example, blue, or pink, or green, or another color. For example, copper phthalocyanine blue can be added to the fibers making up the substrate. A slight tint may give an impression of increased wipe opacity, or may be preferred for aesthetic reasons. In some embodiments, the colorant may be selected to make the wipe appear more white. For example, bluing agents may be used to create a slight blue tint that is perceived not as blue, but as white. An example of a bluing agent is FD&C Blue #1, available from Sensient Cosmetic Technologies Corp. (South Plainfield, N.J.). Alternatively, a colorant may be selected such that the colorant combines with the color of the substrate to create a slight blue tint that is perceived as white.

The colorant or brightener, if present, could also be added to the lotion, or to both the lotion and the substrate. For example, a water soluble dye, such FD&C Green #3 or FD&C Blue #1, or mixtures thereof, can be solubilized into an aqueous lotion. Increased perception of whiteness may be cognitively associated by consumers with increased cleaning effectiveness. The colorant or brightener may coordinate with a retaining agent to improve the retention of the colorant and/or brightening agent on the substrate. In this regard, the discussion of retaining agents with regard to opacifiers applies also to colorants and/or brighteners, if colorants and/or brighteners are used.

Some substrates may be treated such that the substrate has varying density across the length and/or width of the substrate. This is to be distinguished from inherent variation in the density of the substrate, such as normal process variation. For example, some substrates may be hydromolded, embossed, ring-rolled, tintered, or otherwise treated, as discussed above. The treatment may be applied specifically to modify the density of the substrate, or the modification of the density of the substrate may be a secondary effect. For example, a substrate may be embossed with a pattern or design for aesthetic effect, with the result that some portions of the substrate, such as parts of the embossed pattern, are more dense than other portions of the substrate. It may be desirable to use an opacifying lotion as described herein in conjunction with a substrate having varying density. For example, it may be desirable to use an opacifying lotion to exacerbate and/or minimize any differences in opacity related to varying density in the substrate. As a more specific example, in a substrate embossed with a pattern of densified waves, it may be desirable to apply an opacifying lotion to the densified portion of the pattern, to intensify the opacity of the densified portion of the pattern. Alternatively, it may be desirable to apply an opacifying lotion to the undensified or dedensified portion of a pattern, to reduce the difference in the appearance between densified and undensified or dedensified portions. In either case, where the lotion is applied to the densified portion or where the lotion is applied to the undensified or dedensified portions, the lotion application pattern may be considered complementary to the embossed or otherwise formed pattern in the substrate. A complementary lotion application pattern may, for example, be desirable to emphasize, de-emphasize, or otherwise modify the aesthetic effects of aesthetic and/or functional substrate treatments. A complementary lotion application pattern may be identical to a densified or de-densified region, adjacent to a densified or de-densified region, alternating to a densified or de-densified region (e.g., stripes of densification or de-densification with stripes of lotion in between the stripes of densification or de-densification, or concentric with an area of densification or de-densification). Whether or not the lotion application pattern is complementary, the lotion may be applied continuously or intermittently. In particular, the lotion may be applied continuously or intermittently without regard to whether the substrate has been mechanically modified or whether the mechanical modifications to the substrate are continuous or intermittent. In some embodiments, an opacifying lotion applied homogeneously to a substrate having varying density may modify the aesthetic effect of the density variations, as the opacifying lotion and the density variations may combine to make the densified portions more noticeable, relative to the undensified or dedensified portions, than they were before the opacifying lotion was applied.

An opacifying lotion may be selectively applied to a portion of a substrate using a variety of processes, such as printing, spraying, kiss-rolling (with a formed roller), and the like. That is, the opacifying lotion may be applied only to the portion of the substrate that is desirably opacified. No lotion, or a different lotion, including possibly a non-opacifying lotion, may be applied to other portions of the substrate. Selectively applied lotions may be sequestered in the desired location(s) on the substrate using a variety of techniques. For example, immiscible lotions may be applied simultaneously, or in quick succession, to different portions of the substrate, such that the lotions tend to stay where they are applied. The lotion viscosity and/or chemical properties (such as net lotion charge, lotion pH, presence of a retaining aid, type of retaining aid, hydrophilicity, hydrophobicity, surface tension, or combinations thereof) and/or the substrate properties (such as fiber charge, fiber pH, pore size or capillary properties, fiber length, fiber diameter, fiber cross sectional shape, fiber surface morphology, fiber density, fiber strength, fiber splittability, fiber type, fiber hydrophilicity, fiber hydrophobicity, or combinations thereof) may be varied to encourage or discourage interaction between the lotion and certain regions of the substrate. The substrate fibers and/or pore structure may be varied to encourage or discourage the movement and wetting of a lotion through and/or across the substrate. For example, hydrophilic fibers may be used to facilitate movement and wetting of an aqueous lotion along a hydrophilic fiber, and hydrophobic fibers may be used to retard movement and wetting of an aqueous lotion along a hydrophobic fiber. Thus, by varying the physical and/or chemical properties of the substrate and/or lotion, it may be possible to apply the lotion to the substrate more or less homogeneously (i.e., by immersion of the substrate in the lotion), and still obtain a heterogeneous distribution of the lotion on the wipe. That is, the lotion may be loaded onto the wipe heterogeneously via homogeneous application of the lotion. In some embodiments, the lotion may comprise a retaining aid selected to interact with portions of the substrate. For example, portions of the substrate may comprise fibers having a specific chemistry, and the lotion may comprise a retaining aid attracted to the specific chemistry of the specific substrate fibers.

The lotion may be selectively applied to coordinate with the substrate, such as an embossed substrate, as described above, or the lotion may be selectively applied to a homogeneous substrate. An opacifying lotion selectively applied to a homogeneous substrate may be used to create a visible pattern or modify the appearance of a substrate, particularly, but not exclusively, where the opacifying lotion further comprises a brightener, colorant, or other visually observable component, as described above. A lotion may be selectively applied in the x- or y-direction of a wipe substrate (i.e., the length or width of the substrate), as when applied in stripes, dots, or other patterns. A lotion may also be selectively applied in the z-direction of a wipe substrate (i.e., the depth of the substrate). For example, a first lotion may be applied to one surface of a wipe substrate (i.e., the top or first surface), and a second lotion may be applied to the opposing surface of the wipe substrate (i.e., the bottom or second surface). Of course, a lotion may be selectively applied in the x-, y-, and z-directions, and more than one lotion may be selectively applied in any or each direction. In some embodiments, a first lotion is applied homogeneously to the substrate, and a second lotion is selectively applied to the substrate.

A method of opacifying a wet wipe may comprise providing a substrate and loading a lotion onto the substrate. The lotion may comprise a first opacifying agent at a level between 0.1% and 10%, or between 0.1% and 5%, or between 0.1% and 2%, weight of the opacifying agent to weight of the lotion. The substrate may have a basis weight between 20 and 75 gsm. The substrate may comprise fibers. The substrate may comprise a second opacifying agent. The second opacifying agent may be integral to the substrate fibers. The second opacifying agent may be the same as or different than the first opacifying agent in the lotion. The lotion may be loaded onto the substrate at a lotion load between 110% and 600%, weight of the lotion to weight of the unloaded substrate. The lotion may be applied homogeneously or heterogeneously. The lotion may be selectively applied in a pattern or design. The lotion may be applied homogeneously to a substrate, wherein the lotion and the substrate coordinate to sequester the lotion in a pattern or design. The lotion may be a personal cleansing composition. A personal cleansing composition may comprise a surfactant, an emollient, a film-former, particles, preservatives, skin care agents, and/or other additives.

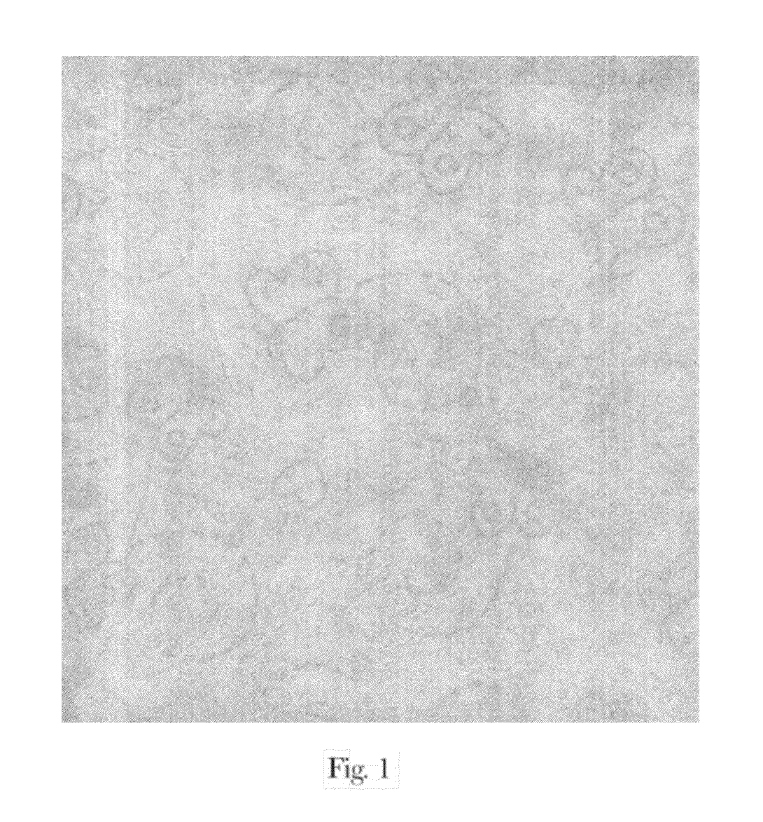

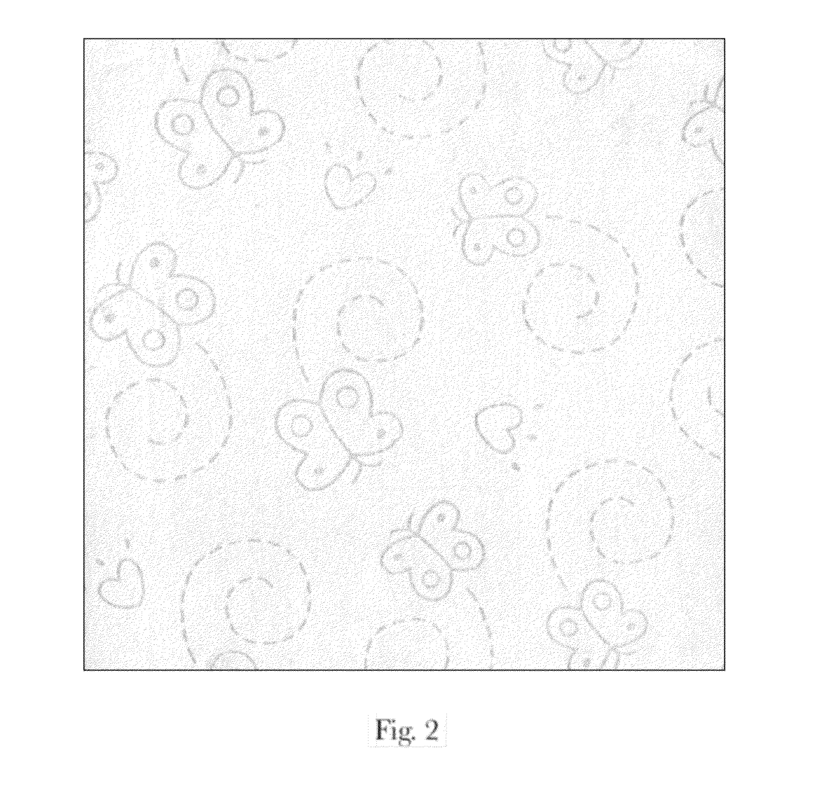

In some embodiments, loading an embossed substrate with an opacifying lotion as described herein may improve the aesthetic effect of the embossing. In some embodiments, the clarity and/or visibility of the embossing on an embossed substrate may be improved by loading an opacifying lotion as described herein onto the substrate. In some aspects, the invention relates to a method for improving the clarity of an embossing pattern on an embossed substrate by applying an opacifying lotion to the substrate. The opacifying lotion may be applied after the substrate has been embossed. FIG. 1 is a scanned image of an exemplary nonwoven substrate loaded with a conventional, aqueous lotion. FIG. 2 is a scanned image of an exemplary nonwoven substrate of the same type (e.g., material and basis weight), loaded with a comparable, aqueous lotion comprising 0.6% TiO₂.

### Examples

Example 1 is an illustrative, non-limiting formula for an opacifying lotion comprising an inorganic opacifying agent.

Example 1

| Ingredient Name | Weight % |
|---|---|
| PEG-40 Hydrogenated Castor Oil | 0.33 |
| Bis-PEG/PPG-16/16 PEG/PPG-16/16 Dimethicone | 0.33 |
| Disodium EDTA | 0.1 |
| Sodium Benzoate | As required for |
| | preservation |
| Xanthan Gum | 0.1 |
| Microcrystalline Cellulose | 0.3 |
| Phenoxyethanol | As required for |
| | preservation |
| Benzyl Alcohol | As required for |
| | preservation |
| Trisodium Citrate | 0.3 |
| Citric Acid | 0.5 |
| Titanium Dioxide | 0.4 |
| Water | 96.92 |

Example 2 is an illustrative, non-limiting formula for an opacifying lotion comprising a polymeric opacifying agent.

Example 2

| Ingredient Name | Weight % |
|---|---|
| PEG-40 Hydrogenated Castor Oil | 0.33 |
| Bis-PEG/PPG-16/16 PEG/PPG-16/16 Dimethicone | 0.33 |
| Disodium EDTA | 0.1 |
| As required for preservation | As required for |
| | preservation |
| Xanthan Gum | 0.1 |
| Microcrystalline Cellulose | 0.3 |
| Phenoxyethanol | As required for |
| | preservation |
| Benzyl Alcohol | As required for |
| | preservation |
| Trisodium Citrate | 0.3 |
| Citric Acid | 0.5 |
| Anionic Styrene/Acrylic Copolymer (Opulyn 301 | 12.6 |
| from Dow Chemical) (40% active) | |
| Water | 84.72 |

Methods

Opacity

Opacity of a nonwoven substrate and/or a wipe comprising a nonwoven substrate is measured using EDANA Recommended Test Method: Nonwovens Opacity 110.1-78 (2002). In executing the test, the sealed or packaged wipe samples are conditioned at 23° C. and 50% relative humidity prior to removing the sealed or packaged wipe for analysis. In step 7.2, samples are cut to a size as dictated by the illumination port of the instrument. Depending on the instrument model, samples as small as 0.5 inches in diameter can be measured. The instrument is calibrated and validated using standardized tiles supplied by Hunter Associates Laboratory (Reston, Va.), or equivalent.

Refractive Index

The Refractive Index for Transparent Organic Plastics can be measured using ASTM Method D542-00 (Reapproved 2006), Standard Test Method for Index of Refraction of Transparent Organic Plastics.

The Refractive Index for Fats and Oils (such as the oleaginous component of an oil-in-water or water-in-oil emulsion) can be measured using American Oil Chemists' Society (AOCS) Official Method Cc 7-25 (Reapproved 2009), Refractive Index.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An aqueous lotion comprising a first, inorganic opacifying agent at a level between 0.1% and 2% weight of the inorganic opacifying agent to weight of the lotion.

2. The aqueous lotion of claim 1, wherein the first, inorganic opacifying agent has a refractive index greater than 1.33.

3. The aqueous lotion of claim 1, wherein the first, inorganic opacifying agent is selected from the group consisting of titanium dioxide, zinc oxide, boron nitride, kaolin clay, calcined kaolin clay, montmorillonite clay, calcined montmorillonite clay, smectite clay, talc, barium sulfate, bentonite clays, silicates including sodium magnesium silicates and fluorosilicates, silicas and surface modified silicas, calcium carbonate and precipitated calcium carbonate, zirconates including strontium zirconate, mica coated with titanium dioxide, mica coated with metal oxides, mica coated with titanium dioxide and other metal oxides, and combinations thereof.

4. The aqueous lotion of claim 1, further comprising a retention aid.

5. The aqueous lotion of claim 4, wherein the retention aid is selected from the group consisting of acrylamide copolymers, polyethylene imines, copolymers of ethylene imine and acrylamide, polyamines, polyethylene oxides, polydiallyldimethylammonium chloride, hydrogenated castor oil, starches, modified starches, guar gums, modified guar gums, celluloses, modified celluloses, silica, bentonite clays, polyaluminum chloride, and combinations thereof.

6. The aqueous lotion of claim 4, wherein the retention aid is present at a level between 0.1% and 20% weight of the retention aid to weight of the lotion.

7. A wet wipe comprising the aqueous lotion of claim 1 and a substrate.

8. The wet wipe of claim 7, wherein the substrate has a basis weight less than 75 gsm.

9. The wet wipe of claim 7, wherein the substrate is a nonwoven and comprises fibers.

10. The wet wipe of claim 7, wherein the substrate comprises a chemical brightener or colorant.

11. The wet wipe of claim 7, wherein the substrate comprises a second opacifying agent.

12. The wet wipe of claim 11, wherein the substrate comprises a binder, and the second opacifying agent is a component of the binder.

13. The wet wipe of claim 11, wherein the second opacifying agent is integral to one or more of the fibers in the nonwoven substrate.

14. The wet wipe of claim 11, wherein the second opacifying agent in the substrate is the same as the first, inorganic opacifying agent in the lotion.

15. A method of opacifying a substrate, the method comprising:
providing a substrate; and
loading an aqueous lotion onto the substrate, the aqueous lotion comprising a first, inorganic opacifying agent at a level between 0.1% and 2% weight of the opacifying agent to weight of the lotion.

16. The method of claim 15 wherein the substrate has a basis weight between 20 and 75 gsm.

17. The method of claim 15 further wherein the substrate comprises a second opacifying agent.

18. The method of claim 17 wherein the first and second opacifying agents are the same.

19. The method of claim 15 wherein the aqueous lotion is loaded on the substrate at a level between 110% and 600%, weight of the aqueous lotion to weight of the unloaded substrate.

20. The method of claim 15, wherein the substrate is a nonwoven substrate having a basis weight between 20 and 75 gsm and the aqueous lotion is a personal cleansing composition.
